(19) United States
(12) Patent Application Publication
(10) Pub. No.: US 2019/0038173 A1
(43) Pub. Date: Feb. 7, 2019

(54) DEVICE AND METHOD FOR DETERMINATION OF DIFFERENCE PARAMETERS ON THE BASIS OF EIT DATA

(71) Applicant: Drägerwerk AG & Co. KGaA, Lubeck (DE)

(72) Inventor: Yvo GÄRBER, Breitenfelde (DE)

(21) Appl. No.: 16/044,973

(22) Filed: Jul. 25, 2018

(30) Foreign Application Priority Data
Aug. 2, 2017 (DE) 10 2017 007 224.8

(51) Int. Cl. A61B 5/053; A61B 5/08; A61B 5/087; A61M 16/01; A61M 16/00; A61B 5/00

(52) U.S. Cl. CPC A61B 5/0536; A61B 5/0809; A61B 5/0871; A61M 16/01; A61M 2230/65; A61B 5/7264; A61B 5/7278; A61M 2230/40; A61M 16/024

(57) ABSTRACT

A device (10) determines difference parameters (300, 400), such as end-expiratory impedance values, on the basis of electrical impedance tomography (EIT) data (36) of regions of the lungs of a living being. The EIT data (36) are obtained by an electrical impedance tomography apparatus (30). A quantitative evaluation of changes in regions of the lungs in terms of hyperdistension or collapsing is provided based on the determined difference parameters (300, 400).

### CROSS REFERENCE TO RELATED APPLICATIONS

This application claims the benefit of priority under 35 U.S.C. § 119 of German Application 10 2017 007 224.8, filed Aug. 2, 2017, the entire contents of which are incorporated herein by reference.

### FIELD OF THE INVENTION

The present invention pertains to a device and a method for electrical impedance tomography with determination of difference parameters on the basis of electrical impedance tomography (EIT) data.

### BACKGROUND OF THE INVENTION

Devices for electrical impedance tomography (EIT) are well known from the state of the art. These devices are configured and intended for generating an image, a plurality of images or a continuous sequence of images from signals obtained by means of electrical impedance measurements and data and data streams obtained therefrom. These images or sequences of images show differences in the conductivity of different tissues of the body, bones, skin, body fluids and organs, especially of the lungs, which are useful for observing the situation of the patient.

Thus, U.S. Pat. No. 6,236,886 describes an electrical impedance tomograph having an array of a plurality of electrodes, power input to at least two electrodes, signal acquisition at the remaining electrodes and a method with an algorithm for image reconstruction for the determination of the distribution of the conductivities of a body, such as bones, skin and blood vessels in a general embodiment with components for signal acquisition (electrodes), signal processing (amplifier, A/D converter), power input (generator, voltage-current converter, current limitation) and control components. U.S. Pat. No. 6,236,886 is incorporated herein by reference in its entirety.

It is stated in U.S. Pat. No. 5,807,251 that it is known in the clinical application of EIT that a group of electrodes is provided as a ring of electrodes, which are arranged at a defined distance from one another, for example, around the chest of a patient in electrical contact with the skin. An electric current signal or voltage input signal is each applied alternatingly between different pairs of electrodes or between all the possible pairs of electrodes among electrodes arranged adjacent to one another. While the input signal is applied to one of the pairs of electrodes arranged adjacent to one another, the currents or voltages are measured between each pair of remaining electrodes, which are adjacent to one another, and the measured data obtained are processed in the known manner in order to obtain a visualization of the distribution of the specific electric resistance over a cross section of the patient, around which the ring of electrodes is arranged, and to display it on a display screen.

Unlike other imaging radiological methods (X-ray apparatuses, radiological computed tomographs), electrical impedance tomography (EIT) has the advantage that no radiation burden that is harmful for the patient occurs. Unlike sonographic methods, continuous image acquisition can be carried out with EIT over a representative cross section of the entire thorax and/or the lungs of the patient by means of the electrode belt. In addition, the need for using a contact gel, which has to be applied before each examination, is eliminated. Electrical impedance tomography (EIT) thus offers the advantage of making a continuous monitoring of the lungs possible in order to observe and document the course of a therapy of a mechanically ventilated or spontaneously breathing patient.

By means of an electrode array around the chest of a patient with an EIT apparatus, as is known, for example, from U.S. Pat. No. 5,807,251, an impedance measurement is carried out on the chest and an image of the lungs of the patient is generated from the impedances by means of a conversion to the geometry of the chest. U.S. Pat. No. 5,807,251 is incorporated by reference herein in its entirety. An EIT device, with a total number of, for example, 16 electrodes, which are arranged in an electrode plane around the chest of a patient, can generate a 32×32-pixel image of the lungs in a circulation of power inputs to two electrodes each and pickup of measured voltage values (EIT measured signals) at the remaining electrodes. In this connection, at the 16 electrodes, a number of 208 measured impedance values is acquired at the electrodes.

These 208 measured impedance values then result in the 1,024 pixels, often also called pixels, using EIT image reconstruction, which pixels then altogether produce the tidal image (TID image) of the lungs in a transverse plane, or transverse view in the region of the electrode plane. The electrodes are arranged in a horizontal array around the thorax of a living being and covering a region of the lungs of the living being for carrying out the electrical impedance tomography (EIT). This results in a position in the plane of the electrode array, which can be designated as a thoracic-axial position of the electrode array on the circumference of the transverse plane of the body.

Electrical impedance tomography (EIT) may advantageously be used for lung function monitoring, for example, and preferably such that a change in the state of a lung compared to a reference is used for monitoring.

So-called dEELI (delta of End Expiratory Lung Impedance) images can consequently be generated by means of electrical impedance tomography (EIT), wherein regional end-expiratory impedances of a time are correlated with those of a reference time. By adding up over all pixels, a value that is in good approximation proportional to an end-expiratory air content change is obtained. This value can then be provided, for example, as an alphanumeric value as additional information on the dEELI image. Furthermore, it is possible to correlate changes in the regional ventilation of the lungs with a reference in order to monitor the state of the lungs. A so-called Differential Tidal image (dTID image) can thus be determined from a difference between a current tidal image and a tidal image of a reference time, i.e., a reference tidal image. A total value of the ventilation change in relation to the reference can be determined from this by adding up the regional differences.

While the ventilation is frequently improved in the entire lung region and a global value of the end-expiratory air content or the change in the end-expiratory air content is often useful for monitoring the ventilation therapy in case of increases in the ventilation pressure in patients with healthy lungs, for example, in case of a PEEP increase, i.e., in case of an increase in the pressure value, which is given at the end of a phase of expiration in the lungs of a patient (PEEP=Positive End Expiratory Pressure), this often does not apply to the ventilation changes especially in diseased lungs. For example, the ventilation in dorsal lung regions, i.e., in lung regions close to the spine, is typically limited when the patient is in the dorsal position. The ventilation here primarily ventilates ventral lung regions, i.e., lung regions close to the sternum of the patient. In case of a PEEP increase as a therapeutic action, the ventilation in the dorsal lung region increases in the ideal case, wherein the compliance thereof improves here, but often at the expense of the compliance in the ventral lung region. A further increase in pressure can then already lead to hyperdistension in the ventral region, while the dorsal region still benefits from the pressure increase with an improvement in compliance. By adding up all dTID pixels, it is thus entirely possible that overall no major ventilation changes can be determined despite differences in the ventilation between the dorsal and ventral lung regions. This does not do justice to strong regional differences and may possibly lead to false interpretations. Ventilation by means of a volume-controlled mode of ventilation should be cited as an example here. Overall no changes in volume between two tidal images as Differential Tidal image (dTID image) are then obtained from the added-up differences, since the tidal volume is kept constant by means of the volume-controlled mode of ventilation. To date, one typically manages with adding up in previously fixed regions, so-called ROIs (regions of interest), in order to minimize this effect, but this often images the regional conditions only insufficiently, and therefore, this solution is not considered to be optimal.

### SUMMARY OF THE INVENTION

With the knowledge of the above-described drawbacks of the known prior art, the object of the present invention is to provide an electrical impedance tomography (EIT) device suitable for imaging the lungs with an electrode array associated with the electrical impedance tomography device, which makes it possible to determine difference parameters of impedance values of lung regions.

Another object of the present invention is to provide a method for the determination of difference parameters of impedance values of lung regions.

According to the invention, a device is provided for determination of difference parameters based on electrical impedance tomography (EIT) data. The device comprises a data input unit, receiving the EIT data obtained by means of an electrical impedance tomography apparatus, wherein the data input unit is configured to receive and provide EIT data from at least one region of the lungs of a living being over an observation period, an output unit and a calculation and control unit connected to the output unit and connected to the data input unit. The calculation and control unit is configured to determine regional impedance values at a first time from the EIT data for at least two regional zones of the lungs, to determine additional regional impedance values at at least one additional time for the at least two regional zones of the lungs, the at least one additional time, as a further time, chronologically succeeding the first time, to determine a regional difference value between the impedance value at the first time and the impedance value at the additional time for each of the at least two regional impedance values, to classify difference values from the regional difference values determined on the basis of an evaluation criterion, and to add up the classified difference values and to determine difference parameters therefrom, which indicate regional property changes in the at least two regional zones of the lungs and to generate and provide a control signal from the difference parameters. The output unit is configured to use the control signal to provide or output an output signal, which represents a numerical value of the regional property changes of the at least two regional zones of the lungs.

According to another aspect of the invention, a method is provided for determination of difference parameters based on electrical impedance tomography (EIT) data. The method comprises providing EIT data associated with a first time and determining first time regional impedance values based on the EIT data associated with the first time and providing the first time regional impedance values as a first time regional impedance values data set. EIT data associated with a further time is also provided and further time regional impedance values are determined based on the EIT data associated with the further time to provide the further time regional impedance values as a further time regional impedance values data set. Regional difference values of ventilated lung regions are determined based on the first time regional impedance values data set and the further time regional impedance values data set. The determined regional difference values are classified based on an evaluation criterion. The classified difference values are added up and difference parameters are determined from totals of the classified difference values. A control signal is generated from the difference parameters.

Furthermore, the method may also be provided as a computer program or as a computer program product, so that the scope of protection of the present application likewise extends to the computer program product and to the computer program.

Of course, features and details, which are described in connection with the method according to the present invention for determination of difference parameters, also apply here in connection with and with respect to the device and vice versa, so that reference is or can always be made alternatingly in relation to the disclosure on the individual aspects of the present invention.

Some of the terms used within the framework of this patent application will be explained in more detail at the beginning.

A time segment in a course over time is defined as an observation period in the sense of the present invention. The beginning and the end of such an observation period are defined either by fixed or adaptable times or by events which are defined by the breathing or ventilation properties. Examples of observation periods, which are based on breathing or ventilation, are a breathing cycle, a plurality of breathing cycles, parts of breathing cycles, such as breathing in (inspiration), inspiratory phase, breathing out (expiration), expiratory phase.

The following signals or data, which can be acquired with an EIT apparatus by means of a group of electrodes or by means of an electrode belt, are defined as measured EIT signals in the sense of the present invention. These include measured EIT signals in different signal characteristic, such as electric voltages or measured voltage signals, electric currents or measured current signals, which are associated with electrodes or groups of electrodes or with positions of electrodes or groups of electrodes on the electrode belt, as well as electric resistance values or impedance values derived from voltages and currents.

A signal feed to two feeding electrodes, to a so-called pair of feeding electrodes, in which measured EIT signals are acquired at other electrodes different from these two feeding electrodes, is defined as a measuring run in the sense of the present invention.

A measurement cycle is defined in the sense of the present invention as a sequence of feeds to a plurality of feeding electrode pairs with a respective corresponding measuring run at the other electrodes. Such a measurement cycle is typically called a so-called “frame” or “time frame” in connection with the processing of EIT data. A number of 208 measured signals is obtained in a measurement cycle, i.e., in a “time frame” in connection with an EIT system with a number of 16 electrodes using an adjacent data acquisition mode.

A measuring run as a part of the measurement cycle is accordingly typically called a “partial frame” in connection with the processing of EIT data. A number of 13 measured signals is obtained in a measuring run, i.e., in a “partial frame” in connection with an EIT system with a number of 16 electrodes using an adjacent data acquisition mode.

The use of the data acquisition mode means that the 13 measured signals are acquired by each of two electrodes positioned adjacent to one another as a measuring electrode pair in a measuring run in connection with feeding to two electrodes positioned adjacent to one another as a feeding electrode pair and then 16 measured signals are obtained in a measurement cycle with rotation of the feeding electrode pair with the 16 measurement cycles for each measuring electrode pair.

An EIT measuring channel is defined in the sense of the present invention as an unambiguous association or constellation of two signal-feeding electrodes and two signal-acquiring electrodes each, which are different from the two signal-feeding electrodes, from a plurality of electrodes. The plurality of electrodes are configured as a component of the electrical impedance tomography device by means of an electrode array, which is configured, for example, as an electrode belt with a defined number of electrodes arranged around the thorax of a patient. Exemplary numbers of electrodes in the electrode belt are 16, 32 or 64 electrodes. There are a plurality of EIT measuring channels, which comprise different associations or constellations of feeding electrodes, on the one hand, and measuring electrodes different therefrom, on the other hand. The EIT measuring channels are preferably addressed in the form of an index-based manner and the data acquired on the EIT measuring channels are preferably addressed in the form of indexed vectors, indexed data fields or indexed matrices, stored and kept ready for further processing (vector operations, matrix operations). The invention advantageously may start when the reconstruction algorithm has already finished creating the an EIT-image, which is stored in a data set (matrix or vector), corresponding to display pixels (pixel values) and based regional impedance values. In case of an EIT system with a number of 16 electrodes with use of an adjacent data acquisition mode, there are 208 EIT measuring channels, wherein one measuring channel each is defined as a clear association of a pair of feeding electrodes and a pair of measuring electrodes. In the adjacent data acquisition mode, two adjacent electrodes of the plurality of electrodes are each used for feeding and two adjacent electrodes of the remaining electrodes from the plurality of electrodes are used for signal acquisition.

Although the invention advantageously uses a data set (matrix or vector) that is provided by a reconstruction algorithm that has already finished producing an EIT-image (pixel data), namely the EIT device does all of the data pre-processing, the invention can also begin with raw data (voltages) as the EIT-Data. Additionally, besides the invention using pixel/impedance value data as the EIT-Data, the device, system and method of the invention may start with already classified pixel data. For example, such a classification of pixel data could be “Not ventilated regions of the lung”, “Insufficient ventilated regions of the lung”, “Sufficient ventilated regions of the lung”, with this data of the three classification types being used as the EIT-Data. As used herein the term pixel data is essentially the same as pixel values, EIT data, impedances, impedance differences or impedance changes. Further, pixel data relating to pixel status is used herein as essentially the same as a lung tissue state, namely ventilated/non ventilated regions of lung tissue. EIT data are defined in the sense of the present invention as the following signals or data:
Raw EIT data, i.e., measured signals acquired with an EIT apparatus by means of a group of electrodes or by means of an electrode belt, such as voltages or currents, associated with electrodes or groups of electrodes or with positions of electrodes or of groups of electrodes on the electrode belt,
EIT image data, i.e., data or signals which were determined from the raw EIT data with a reconstruction algorithm and indicate impedances, impedance differences or impedance changes of the regions of the lungs,
classified EIT data, i.e., EIT image data or signals, which are presorted or preclassified according to predefined criteria. The classification may be configured here, for example, as a division into ventilation-related and into heart-related and perfusion-related EIT data or signals.

According to the present invention:
regional difference values of ventilated lung regions are each determined on the basis of EIT data of a first time and on the basis of EIT data of at least one additional time;
the determined regional difference values are classified on the basis of an evaluation criterion,
the classified difference values are added up,
difference parameters, which indicate regional property changes in at least two regional zones of the lungs, are determined on the basis of the totals of the classified difference values, and
a control signal is generated and provided from the difference parameters.

The regional difference values are preferably classified into positive and negative difference values and all positively classified difference values of the differential tidal image are added up to form a positive difference parameter and all negatively classified difference values of the differential tidal image are added up to form a negative difference parameter.

In this connection, the negative difference parameter represents a loss of ventilation of lung regions and the positive difference parameter represents a win (gain) of ventilation of lung regions.

The object is accomplished by a device according to the present invention according to a first aspect. The device according to the present invention is configured for determination of difference parameters on the basis of EIT data and has as essential components

a data input unit,

a calculation and control unit and

an output unit.

The EIT data are obtained according to the invention by means of an electrical impedance tomography apparatus and are provided to the device for determination of difference parameters directly by the electrical impedance tomography apparatus (in real time) or indirectly by means of data lines, signal lines or network connections and may be provided based on memory stored EIT data that was obtained in advance. The device may also be configured for determination of difference parameters as a component of the electrical impedance tomography apparatus, or the device for determination of difference parameters may be, as it were, associated or coordinated with the electrical impedance tomography apparatus as a complementary element. In addition, the electrical impedance tomography apparatus may interact together with a ventilator or anesthesia apparatus and thus be configured as a medical system.

The data input unit is configured to receive the EIT data from at least one region of the lungs of a living being over an observation period and to provide same to the calculation and control unit. As described before, 208 measured impedance values are acquired with an array of 16 electrodes, from which 1,204 pixels are then generated by means of EIT image reconstruction, e.g., reconstruction (generation of pixel data—pixels) according to U.S. Pat. No. 6,236,886, which can then be provided as the EIT data to the data input unit for further processing in the calculation and control unit.

The calculation and control unit is configured to determine (including select, receive or calculate) regional impedance values from the EIT data. Determining the impedance values from the EIT data may simply comprise receiving of the pre-processed data, for example an EIT-data set from an EIT System with a structure of pixels, each pixel having a certain point in a coordinate system (Cartesian or Polar) in the two-dimensional horizontal layer (transverse view, see of the thorax) and a certain point in time (t1). In this example there is no special data processing needed and involves relocating the information, related to the coordinate system, into regional impedances. In another way the calculation and control unit is configured to determine based on the EIT-data set containing an amount of data, for example a large matrix Z1, X1, Y1 . . . Zn, Xn, Yn with pixels and associated time information t1 . . . tn over a time period of monitoring a patient while changing the PEEP step by step. In this case determining comprises an extracting of the two certain data sets and particular regional impedances are selected to comprise outputs. These regional impedance values indicate the ventilation of a plurality of lung regions, in the form of pixels, for the plurality of pixels (a region). These regional impedance values are stored as a data quantity typically as a data set in a memory or memory area associated with the calculation and control unit and kept ready for the further data processing in this memory or memory area.

The calculation and control unit is configured to determine or receive regional impedance values from the EIT data at a first time t₁ for at least two regional zones of the lungs. The calculation and control unit is configured to determine or receive additional regional impedance values from the EIT data at at least one additional time (further time) tₙ for the at least two regional zones of the lungs. The further time tₙ chronologically follows the first time t₁.

The calculation and control unit is configured to determine a regional difference value between the impedance value at the first time t₁ and the impedance value at the additional time tₙ for each of the at least two regional impedance values and to classify the difference values from the regional difference values determined in at least two state classifications based on an evaluation criterion.

The calculation and control unit is further configured to add up the classified regional difference values in each of the at least two state classifications and to determine difference parameters therefrom, which indicate regional property changes in the at least two regional zones of the lungs.

The calculation and control unit is further configured to generate and provide a control signal from the difference parameters.

The evaluation criterion is preferably applied by the calculation and control unit for a classification of the determined regional difference values in the at least two state classifications such that the regional difference values are distinguished into positive and negative difference values. Positive difference values are obtained for pixels of the tidal image if the regional impedance value at the time tₙ is increased by a first predefined value compared to the regional impedance value at the time t₁. Negative difference values are obtained for pixels of the tidal image if the regional impedance value at the time tₙ is lower by a second predefined value compared to the regional impedance value at the time t₁. Here, for example, threshold values can be used as predefined values; a useful threshold value is, for example, a difference in amount in a range of 5%-15% with reference to the regional impedance value at the time t₁. Positive difference values indicate that the ventilation situation in the lung region corresponding to this pixel (corresponding to a particular lung location) has improved, negative difference values indicate that the ventilation situation in the lung region corresponding to this pixel has worsened.

The calculation and control unit is further configured to carry out an adding up of all negatively classified difference values to form a negative difference parameter (LOSS) and an adding up of all positively classified difference values to form a positive difference parameter (WIN).

The first and second threshold values may be based here on a similar threshold; however, the first and second threshold values may also be configured as different from one another, so that the result is an asymmetric classification for the negative difference parameter (LOSS) and positive difference parameter (WIN).

In this connection, the negative difference parameter (LOSS) indicates regions of the lung, in which the ventilation situation at the time tₙ has worsened under the ventilation therapy compared to the ventilation situation at the time t₁.

In this connection, the positive difference parameter (WIN) indicates regions of the lung, in which the ventilation situation at the time tₙ has improved under the ventilation therapy compared to the ventilation situation at the time t₁.

In this way, it can be shown, for example, when and in what manner the ventilation situation of the dorsal lung regions has improved (WIN) due to a change in a ventilation pressure, for example, in the inspiration pressure or in the pressure, which is given at the end of a phase of expiration in the lungs of a patient (PEEP=Positive End Expiratory Pressure), and whether this improvement in the dorsal lung regions is accompanied by a worsening (LOSS) of other regions, for example, ventral regions.

The difference parameters (WIN, LOSS) are preferably determined by the calculation and control unit as numerical values, for example, as percentage values, which indicate the difference between the ventilation situations of the lung regions at the times tₙ and t₁ in order to make it possible to visualize the regional property changes in the at least two regional zones in a very clear manner in this way.

An improvement or a worsening of the ventilation situation of the lungs can occur due to changes or adaptations of settings at the ventilator, especially due to increases or decreases in the ventilation pressure. However, repositionings of the patient, for example, changing from a dorsal position to a lateral position and vice versa may also bring about an improvement or a worsening of the ventilation situation of the lungs regardless of changes or adaptations of settings at the ventilator or anesthesia apparatus or in combination with changes of adaptations of settings at the ventilator or anesthesia apparatus.

The output unit is configured, using the control signal, to generate, provide or output an output signal, which represents a numerical value of the regional property changes of the at least two regional zones of the lungs.

The regional properties and property changes determined on the basis of the EIT data and regional impedance values represent regional changes in the compliance or elasticity of the lungs.

The numerical value outputted or provided by the output unit may be configured here, for example, as a percentage value, which indicates regional changes in the determined properties, especially the compliance or the elasticity of the lungs compared to a reference variable, preferably a reference variable determined at the beginning of the observation period.

It shall be shown in an exemplary manner by means of the following definitions and formulas and the explanations corresponding to these formulas how changes in the ventilation states “WIN” and “LOSS” can be determined by the calculation and control unit.

A first tidal image is to be defined as a reference tidal image with pixel values Rᵢ, I∈Ω as a quantity of pixels at the time t₁ and a second tidal image with pixel values Tᵢ, I∈Ω as an additional quantity of pixels at the time tₙ in a quantity of pixels in a ventilated region Ω of the lungs of a patient. The reference tidal image is subtracted from the second tidal image by pixels, so that a differential tidal image (dTID image)

Dᵢ=c(Tᵢ,−Rᵢ),I∈Ω

is obtained as a result. The reference tidal image is preferably scaled to 100% by the scale factor c according to Formula 1.

$$c {Σ}_{i ∈ Ω} {R}_{i} = 100 \% \to c = \frac{100 \%}{{Σ}_{i ∈ Ω} {R}_{i}} . \qquad Formula 1$$

In a special embodiment, a scaling or calibration to a tidal volume or a compliance according to Formula 2 can be carried out for the reference tidal image and for each newly determined tidal image and also for the differential tidal image (dTID image). In this connection, the tidal volume as well as compliance, in addition to the values of the ventilation pressure course in the data exchange are each currently made available by a ventilator or anesthesia apparatus to the device for electrical impedance tomography with determination of difference parameters on the basis of EIT data.

$${c}_{R} = \frac{{G}_{R}}{{Σ}_{i ∈ Ω } {R}_{i}} , {c}_{r} = \frac{{G}_{T}}{{Σ}_{i ∈ Ω} {T}_{i}} , {D}_{i} = {c}_{T} {T}_{i} - {c}_{R} {R}_{i} , i ∈ Ω . \qquad Formula 2$$

The symbols G_{R} and G_{T} denote the ventilation observables corresponding to T and R, for instance, tidal volume or compliance, which shall be scaled. No scaling is done in the simplest case. The scale factor would be c=1 in the simplest case.

Two regions should be defined in the quantity of pixels in the ventilated region SZ of the lungs of a patient:

Ω+ is defined as the region of positive difference values Ω+: I∈Ω with Dᵢ>0,

Ω− is defined as the region of negative difference values Ω−: I∈Ω with Dᵢ<0.

The difference parameters W (WIN), W=Σ_{I∈Ω+} Dᵢ and L (LOSS), L=Σ_{I∈Ω-} Dᵢ are then obtained by means of the two regions Ω+, Ω−, by applying the scaling c as numerical percentage values, which indicate the difference between the ventilation situations in the ventilated region Ω of the lungs of a patient at the times tₙ and t₁.

The difference parameters make possible a balancing of regions with “WIN” and “LOSS” and thus advantageously simplify the decision-making for the user, as can then be further proceeded in the therapy of patients. The advantage of the difference parameters is also manifested in situations, in which changes in the medication of the patient are made or were made by the user during the course of the treatment. Effects, which possibly arise due to the manner, for example, the quantity and frequency of a dosage of a certain medication, with regard to the ventilation of the lungs of the patient without changes in the ventilation therapy, are observable over the short term and over the long term (trend analysis) by means of difference parameters.

Thus, for example, changes in the positioning of the patient from the dorsal position into the prone position or lateral position, an additional support in the shoulder region in the dorsal position as well as changes in ventilation settings in ventilation pressure, ventilation rate and inspiration/expiration time ratio contribute to balancing out the “WIN” and “LOSS” and thus to improve the situation for the patient.

The data input unit is configured in a preferred embodiment to receive data, which indicate the measured pressure values or a pressure course of a ventilation pressure in the observation period and to provide said data to the calculation and control unit. The calculation and control unit in this preferred embodiment is configured to determine, on the basis of data, which indicate the measured pressure values or the pressure course of the ventilation course, values in the observation period, which indicate end-expiratory values of a ventilation pressure (PEEP). Should the ventilation situation be characterized for different ventilation pressures by means of the difference parameters W (WIN) and L (LOSS), then it is especially advantageous when the ventilation pressures at the times tₙ and t₁ are different from one another. As was explained in other embodiments, suitable ventilation maneuvers (PEEP trials) may be provided for this in order to let different end-expiratory values of the ventilation pressure (PEEP) in the lungs of the living being take effect for the ventilation at different times of acquisition of the EIT data.

In another preferred embodiment, the calculation and control unit is configured, taking into account the values that indicate end-expiratory values of the ventilation pressure, to determine regional end-expiratory impedance values of the regional zones of the lungs from the regional impedance values, to determine the regional difference values from the regional end-expiratory impedance values and to determine the respective difference parameters, which indicate regional changes in the determined properties, especially the compliance or elasticity of the lungs. In this connection, the difference parameters preferably indicate each a numerical percentage win/loss situation of the compliance in the at least two regions of the lungs. The concept of a win/loss analysis makes it possible to indicate how the regional compliance has changed for regional zones of the lungs with a change in the ventilation pressure.

For this, the calculation and control unit analyzes the pressure course of the ventilation pressure in the observation period such that chronologically corresponding regional end-expiratory impedance values at respective pressure values (PEEP) at the end of respective expiration phases can be used by the calculation and control unit for the determination of the difference values of the pixels and the determination of the difference parameters. It can be determined with this win/loss analysis whether, for example, an increase in ventilation pressure, especially the end-expiratory ventilation pressure (PEEP), in a region of the lungs has led to an opening of previously closed (collapse) regions of the alveoli, which would be evaluated as a “WIN.” If regions with previously already opened alveoli would experience a hyperdistension in the course of the same pressure increase, this would be evaluated as a “LOSS.” In addition, it is also possible to determine whether, for example, a decrease in the ventilation pressure, especially in the end-expiratory ventilation pressure (PEEP), in a region of the lungs has led to a reduction of previously hyperdistended regions of the alveoli, which would be evaluated as a “WIN.” If alveoli would then collapse in regions with previously already opened alveoli in the course of the same decrease in pressure, this would be evaluated as a “LOSS.” The pressure parameters thus reveal information about whether an opening (WIN) of previously closed lung regions has occurred in connection with an increase in pressure at the expense (LOSS) of an increase in hyperdistended lung regions, as well as whether a relief (WIN) of hyperdistended lung regions has occurred in connection with a decrease in pressure at the expense (LOSS) of an increase in closed lung regions. The difference parameters as numerical numbers or percentages reveal in this connection information about the current time tₙ—with the value of the ventilation pressure corresponding to the time tₙ—as are the quantitative changes in the respective surfaces of the lung regions with WIN and LOSS each in comparison to the time t₁—with the value of the ventilation pressure corresponding to the time t₁. For example, it should be noted here that alveoli in the front (ventral) region of the lungs, which are usually closed in the dorsal position of a patient, are better caused to open due to an increase in the ventilation pressure than closed alveoli in the rear (dorsal) region of the lungs, because the weight forces of the organs and tissues lying above them have an effect on the alveoli. As a result, a ventilation pressure adapted in the front regions of the lungs for opening alveoli in rear regions of the lungs could already lead to hyperdistension.

In another preferred embodiment, the duration of at least two breathing cycles with an inspiration period and an expiration period each is selected as the observation period.

A time period of a maneuver with changes in the ventilation pressure, preferably in a decremental PEEP trial with a plurality of breathing cycles, each with an inspiration phase and an expiration phase, is selected as the observation period in another preferred embodiment. The end-expiratory ventilation pressure (PEEP) corresponding to the regional end-expiratory impedance values is in this case lower by a predefined value at the time tₙ compared to the end-expiratory ventilation pressure (PEEP) at the time t₁. For example, a pressure difference of 1 mbar, 2 mbar or 5 mbar may be configured as a predefined value for a lowering level of the end-expiratory ventilation pressure (PEEP). A decremental PEEP trial may, for example, be embodied as a sequence of reductions in the end-expiratory ventilation pressure (PEEP) from a starting value of, for example, 15 mbar to a target value of 5 mbar via a multistep reduction by means of a sequence of lowering levels of 2 mbar.

A time period of a maneuver with changes in the ventilation pressure, preferably in an incremental PEEP trial with a plurality of breathing cycles, each with an inspiration phase and an expiration phase is selected as the observation period in another preferred embodiment. The end-expiratory ventilation pressure (PEEP) corresponding to the regional end-expiratory impedance values is in this case increased by a predefined value at the time tₙ compared to the end-expiratory ventilation pressure (PEEP) at the time t₁. For example, a pressure difference of 1 mbar, 2 mbar or 5 mbar may be configured as a predefined value for an increasing level of the end-expiratory ventilation pressure (PEEP). An incremental PEEP trial may, for example, be configured as a sequence of increases of the end-expiratory ventilation pressure (PEEP) from a starting value of, for example, 6 mbar to a target value of 16 mbar via a multistep increase by means of a sequence of increasing levels of 2 mbar.

At least one rear region, especially a so-called dorsal region, of the lungs is also included in a region of the at least two regional zones of the lungs in another preferred embodiment. Here, regional end-expiratory impedance values of the rear region of the lungs are determined by the calculation and control unit from the regional impedance values, the regional difference values are determined subsequently by the calculation and control unit from the end-expiratory impedance values of the rear region of the lungs, from which regional difference values the difference parameters, which indicate the property changes in the rear, dorsal region of the lungs, are determined by the calculation and control unit.

In another preferred embodiment, at least one front region, especially a so-called ventral region, of the lungs is also included in a region of the at least two regional zones of the lungs. In this case, regional end-expiratory impedance values of the front region of the lungs are determined by the calculation and control unit from the regional impedance values, the regional difference values are subsequently determined by the calculation and control unit from the end-expiratory impedance values of the front region of the lungs, from which regional difference values the difference parameters, which indicate the property changes in the front, ventral region of the lungs, are determined by the calculation and control unit.

In another preferred embodiment, the calculation and control unit is configured to determine the property changes in the ventral region of the lungs and the property changes in the dorsal region of the lungs each as difference parameters and to generate a control signal and to provide an output unit, which indicates the differences in the change in the compliance as difference parameters as a function of a pressure-time course or of a time course of an incremental or decremental PEEP trial.

The output unit is preferably configured, using the control signal, to generate, provide or output an output signal, which represents the difference parameters as a function of the pressure-time course and/or of the time course of the incremental or decremental PEEP trial and/or as numerical win/loss situations, preferably as numerical values, of the regional changes in the compliance in the ventral region and in the dorsal region of the lungs. In this connection, the difference parameters indicate numerical, preferably respective percentage WIN/LOSS situations in the compliance in the ventral region and dorsal region of the lungs.

In another preferred embodiment, the data which indicate a pressure course of a ventilation pressure in the observation period are provided by a ventilator or anesthesia apparatus to the device, inputted and provided by the data input unit and also processed by the calculation and control unit for determination of the difference parameters.

In another preferred embodiment, the device is configured as an apparatus or as a combination of apparatuses for determination of the difference parameters to be determined. Such an apparatus or such a combination of apparatuses has, for example, functions for determination and visualization of properties of the lungs or property changes of the lungs, as well as functions for determination of the difference parameters. Such an apparatus or such a combination of apparatuses has in another preferred embodiment, for example, functions for carrying out a ventilation and/or carrying out an anesthesia and, for example, in addition, functions for carrying out an impedance tomography (EIT) with determination and visualization of properties of the lungs or property changes of the lungs and determination of the difference parameters.

The present invention was described above according to a first aspect of the present invention for the device according to the present invention for determination of difference parameters on the basis of EIT data. According to another aspect, a method according to the present invention is provided for determination of difference parameters on the basis of EIT data. The method is structured in the form of a sequence of steps. Other embodiments of the individual steps, divisions into single steps or combinations of steps or arrangement of the order of the sequence of steps are also covered by the inventive idea for the determination of difference parameters on the basis of EIT data. In the sequence of steps
EIT data, which are provided and associated with a first time, are inputted in a first step,
regional impedance values are each determined on the basis of the EIT data associated with the first time and are provided as a data set in a second step,
EIT data provided and associated with an additional time are inputted in a third step,
regional impedance values are each determined on the basis of the EIT data associated with the additional time and are provided as a data set in a fourth step,
regional difference values of ventilated lung regions are each determined on the basis of the data sets with the determined regional impedance values and are classified on the basis of an evaluation criterion in a fifth step,
the classified difference values are added up and the difference parameters are determined from the totals of the classified difference values in a sixth step, and
a control signal is generated and provided from the difference parameters in a seventh step.

In a preferred embodiment of the method, additional EIT data are inputted or provided in the third step, for example, in partial steps corresponding to the third step and processed to form additional difference parameters L, W. Additional current tidal images are determined during the course of the processing in the following steps, so that determination of a series of differential tidal images is thus also possible and determination of a series of difference parameters is thus also possible.

In a preferred embodiment of the method, the additional difference parameters are determined in connection with a change in pressure situations in the lungs of a patient.

In another preferred embodiment of the method, the change in pressure situations is configured as a gradual change in an expiratory pressure (PEEP=Positive End Expiratory Pressure) at the end of the respective expiration phase. This gradual change in the expiratory pressure (PEEP=Positive End Expiratory Pressure) may, for example, be caused by means of a maneuver carried out by a ventilator. Such a maneuver is configured, for example, as an incremental or decremental PEEP trial, in which the positive end-expiratory pressure (PEEP) is gradually increased or decreased from a starting value.

Each of the embodiments described represents, in itself as well as combined with each other, special configurations of the device according to the present invention for the determination of difference parameters. In this case, advantages arising due to a combination or combinations of a plurality of embodiments and other embodiments are nevertheless also covered by the inventive idea, even if not all combination possibilities of embodiments are each explained in detail for this purpose. The advantages described for the method according to the present invention can be achieved in the same manner or in a similar manner with the device according to the present invention as well as with a device for carrying out the method according to the present invention, as well as the described embodiments of the device. Furthermore, the described embodiments and the features and advantages thereof of the method are applicable to the device, and the described embodiments of the device are applicable to the method. The corresponding functional features of the method are formed here by corresponding objective modules of a device, especially by hardware components (μ, DSP, MP, FPGA, ASIC, GAL), which can be implemented, for example, in the form of a processor, a plurality of processors (μ, μP, DSP) or in the form of instructions in a memory area, which instructions are processed by the one or more processor. The above-described embodiments of the method according to the present invention may also be configured in the form of a computer-implemented method as a computer program product with a computer, wherein the computer is prompted to execute the above-described method according to the present invention, when the computer program is executed on the computer or on a processor of the computer or a so-called “embedded system” as part of a medical device, especially of the EIT apparatus. In this case, the computer program may also be stored on a machine-readable memory medium. In an alternative embodiment, a memory medium may be provided, which is intended for the storage of the above-described, computer-implemented method and can be read by a computer. It lies within the framework of the present invention that not all steps of the method absolutely have to be executed on one and the same computer, but rather they may also be executed on different computers, for example, in a form of Cloud Computing previously described in more detail. The sequence of the method steps may also possibly be varied. Furthermore, it is possible that individual sections of the above-described method may be executed in a separate unit, which may, for example, be sold separately (such as, e.g., on a data analysis system preferably arranged in the vicinity of the patient), other parts on a different unit capable of being sold (such as, e.g., on a display and visualization unit), which is preferably arranged, for example, as a part of a hospital information system, set up in a room for the monitoring of a plurality of patient rooms, as it were, can be executed as a distributed system.

The present invention will now be explained in more detail by means of he following figures and the corresponding figure descriptions without limitations of the general inventive idea. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view of a differential tidal image;

FIG. 2 is a graphic diagram of difference parameters at different pressure levels of the end-expiratory pressure;

FIG. 3 is a schematic overview of an electrical impedance tomography apparatus; and

FIG. 4 is a schematic view of a flow chart for determination of difference parameters.

### DESCRIPTION OF THE PREFERRED EMBODIMENTS

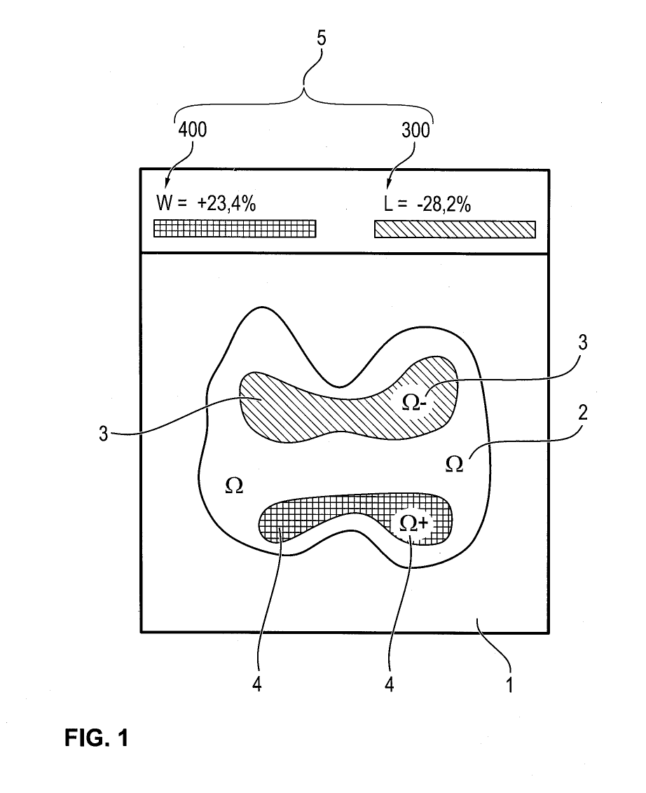

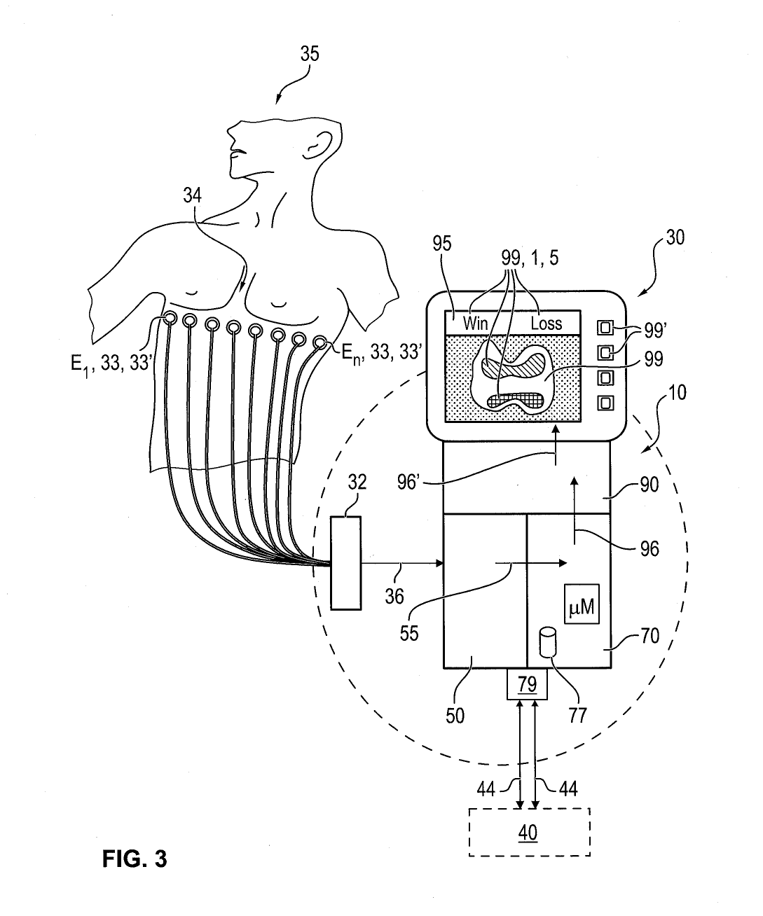

Referring to the drawings, FIG. 1 shows a schematic view of a differential tidal image 1 (dTID image). A ventilated lung region Ω2, a classified lung region Ω+ 4, whose state of ventilation is indicated by a difference parameter W 400 characterizing an improvement (WIN) of the ventilation, and an additional classified lung region Ω−3, whose state of ventilation is indicated by a difference parameter L 300 characterizing a worsening (LOSS) of the ventilation, are shown. The calculation of the difference parameters W 400 and L 300 is carried out such that a reference tidal image with pixel values Rᵢ, I∈Ω is acquired as a quantity of pixels at a first time and a current tidal image with pixel values Tᵢ, I∈Ω is acquired as a quantity of pixels at an additional time in relation to impedance values of the lungs and/or regions of the lungs derived from EIT data 36 (FIG. 3). A value of a pixel, is for example an impedance value or simply a status, like “Ventilated”/“Not Ventilated”. In this example being shown in this FIG. 1, the so-called positive end-expiratory pressure, i.e., the pressure level (PEEP) present in the lungs should be increased by a predefined value, for example, 10 mbar after complete expiration of the patient in connection with acquisition of the current tidal image compared to the pressure level in connection with the acquisition of the reference tidal image. Subsequently, the reference tidal image in the form of pixels (pixel data values) is subtracted from the second tidal image in the form of pixels (pixel data values), so that a differential tidal image 1 (dTID image) Dᵢ=(Tᵢ−Rᵢ), I∈Ω is obtained as a result. In the differential tidal image 1, the lung regions Ω2 are classified into lung regions Ω− 3 and Ω+ 4. Ω+ is defined here as the region of positive difference values Ω−: I∈Ω with Dᵢ>0; Ω− is defined here as the region of negative difference values Ω−: I∈Ω with Dᵢ<0. By means of the two regions Ω− 3 and Ω+ 4, the difference parameters W=Σ_{I∈Ω+} Dᵢ 400 (WIN) and L=Σ_{I∈Ω-} Dᵢ 300 (LOSS) are then obtained as numerical percentages, which indicate the difference between the ventilation situations in the ventilated region Ω 2 of the lungs of a patient between two or more different times of ventilation. The difference parameters 300, 400 are displayed in FIG. 1, for example, by means of a numerical output field 5 above the differential tidal image 1. The classified lung regions Ω− 3, Ω+ 4 are shown in this FIG. 1 in an exemplary manner as respective, connecting lung regions, wherein the classified lung region Ω− 3 corresponds to rear (dorsal) regions of the lungs and the classified lung region Ω+ 4 corresponds to front (ventral) regions of the lungs.

Difference parameters W=23.4% (WIN) and L=−28.2% (Loss) 300 are obtained in this example shown in this FIG. 1. It can hence be concluded that the ventilation has improved by 23.4% of the total value in comparison to the reference tidal image in the dorsal region, whereas the ventilation in the ventral region has worsened by −28.2%. There is a significant difference between the dorsal region and the central region of more than 50%. If a single global value is indicated for the dTID image, it could only be concluded that the ventilation has overall only changed by less than 5%, has worsened, on average, by 4.8% in this example. The separate determination and visualization of the difference parameters W 300, L 400 for regions Ω+ 4 (WIN) and Ω− 3 (LOSS) shows, however, that this is not the case and the change in the positive end-expiratory pressure had a great and regionally different effect on the change in lung function. In this example, an improvement in the ventilation in the rear (dorsal) lung regions at the expense of a worsening of the ventilation in front (ventral) lung regions was thus bought about with a ventilation maneuvers, due to the PEEP increase by a predefined value, e.g., 10 mbar. This is very clearly evident by means of the output of the difference parameters W=23.4% (WIN) 400 and L=−28.2% (Loss) 300 and visualization of the classified lung regions Ω− 3, Ω+ 4. The differentiated output by means of the difference parameters W=23.4% (WIN) 400 and L=−28.2% (Loss) 300 and of the corresponding display or visualization of the lung regions Ω 2, Ω− 3, Ω+ 4 is a major advantage for deriving other therapeutic measures, for example, with respect to adaptations of ventilation parameters respiration rate (RR), inspiration to expiration ratio (I:E ratio), tidal volume (Vt), inspiration pressure (Pinsp), expiration pressure (Pexp, PEEP) at the ventilator 40 (FIG. 3) or even nursing measures such as positioning or position changes of the patient (lateral position, dorsal position). The visualization of the lung regions Ω 2, Ω− 3, Ω+ 4 can preferably be color coded or be coded in different gray scales. One advantageous visualization is, for example, a visualization of the Ω− 3 in yellowish shades and a visualization of the Ω+ 4 in bluish shades against a background of the Ω 2 in a neutral color, for example, in dark gray or black.

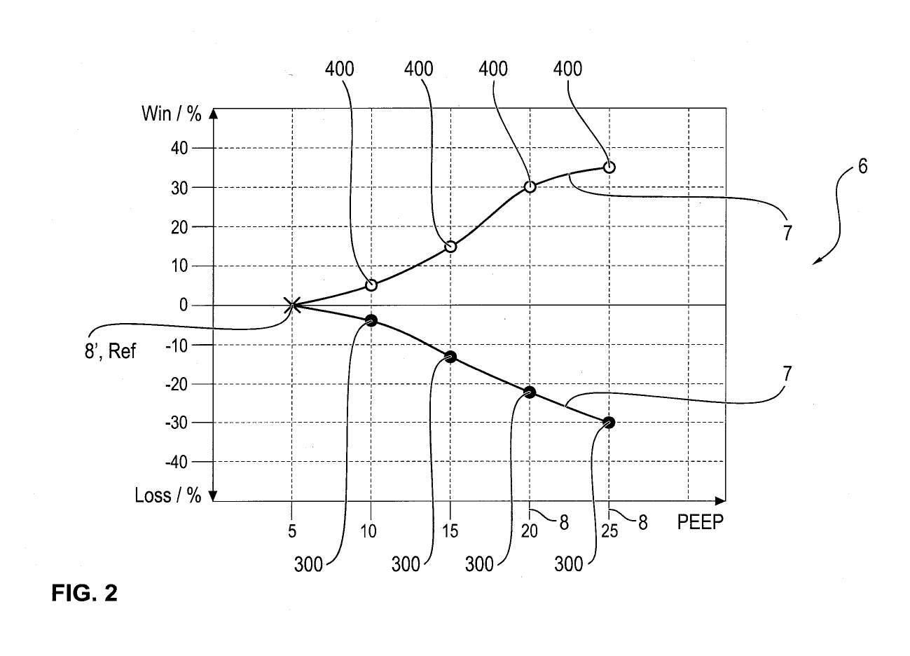

FIG. 2 shows a graphic diagram of a course 7 of difference parameters 300, 400 at different pressure levels 8 of a positive end-expiratory pressure (PEEP). The positive end-expiratory pressure (PEEP) is increased by means of an incremental PEEP trial from a starting value of 5 mbar in 5 steps of 2 mbar, for example, over the course of time to a value of 25 mbar, and the respective WIN/LOSS situation, which is determined from the impedance values of regions Ω+ with positive difference values and of regions Ω− ith negative difference values for the different pressure levels 8, is shown with scaling to the starting value at the pressure level of 5 mbar as a reference pressure level 8′. The graphic diagram 6 shows that some lung regions benefit from the increase in the positive end-expiratory pressure (PEEP), but other lung regions do not benefit. It is also evident that the WIN/LOSS situation for different pressure levels 8 is shown differently. By means of this graphic diagram 6 of the course 7 of difference parameters 300, 400 at the different pressure levels 8, the user is enabled to select a pressure level 8, which is satisfactory in terms of the WIN/LOSS situation with respect to the therapy of the patient 35 (FIG. 3).

FIG. 3 shows a schematic view of an arrangement 10 of an EIT apparatus 30 with electrode array 33 with a plurality of electrodes E1, . . . , En 33′. The electrode array 33 with the electrodes E1, . . . , En 33′ is arranged on the torso (thorax) of a patient 35. A measured value acquisition and feed unit 32 is configured to feed a signal, preferably an alternating current (power input) or even an alternating voltage (voltage feed) to a pair of electrodes 33′ each in a measurement cycle. The voltage signals resulting due to the alternating power input (power input) are acquired as signals at the remaining electrodes 33′ of the measured value acquisition and feed unit 32 and provided as EIT data 36 to the data input unit 50. The provided EIT data 36 are fed as a data signal 55 to a control unit 70 in the EIT apparatus 30 via a data input unit 50. A memory 77, which is configured for a storage of a program code, is provided in the control unit 70. The sequence of the program code is coordinated by a microcontroller arranged in the control unit as an essential element or by other configurations of calculation elements (FPGA, ASIC, μP, μC, GAL). The calculation and control unit 70 is thus configured and intended to determine one or more differential tidal images (dTID) 1 (FIG. 1) from the EIT data 36 and to classify lung regions on the basis of the differential tidal images 1 (FIG. 1) and then to determine and provide difference parameters 5, 300, 400 (FIG. 1) on the basis of the classification as explained previously in the explanations regarding FIG. 1 and in the specification section of the application text. The differential tidal images 1 (FIG. 1) determined and provided by the control unit 70 and ventilated lung regions 2 Ω shown in the differential tidal images (FIG. 1), the lung regions Ω+ 4 (FIG. 1), Ω− 3 (FIG. 1) classified in the lung regions 2 (FIG. 1) and/or difference parameters 300, 400 (FIG. 1) corresponding to the classified lung regions Ω+ 4 (FIG. 1), Ω− 3 (FIG. 1) are provided by means of a data output unit 90 as data signals or control signals 96 to a data output unit 90 and provided by this data output unit 90 and visualized 99 onto a display device 95 by means of control signals 96′. In addition to the visualization 99, additional elements 99′ are present on the display device 95, for example, operating elements (keyboard, switch) or visual indicator elements (LED). The visualization 99 with the additional elements 99′ is configured as a user interface, more preferably as a graphic user interface (GUI), for example, as a so-called touchscreen display. The data input unit 50 and/or the control unit 70 are additionally configured in an optional embodiment to receive data 44 from a ventilator or anesthesia apparatus 40 by means of an interface 79. These data of the ventilator 40 indicate, for example, settings of the ventilator 40, such as respiration rate (RR), inspiration to expiration ratio (I:E ratio), tidal volume (Vt), data, which indicate and/or specify an observation period, such as phases or times of the ventilation as well as data acquired or determined from sensory components of the ventilator 40, signals, measured values or parameters such as inspiratory, expiratory flow values (\dot{V}ᵢₙₛₚ, \dot{V}ₑₓₚ), inspiratory, expiratory pressure values (Pinsp, Pexp, PEEP), inspiratory, expiratory volumes (Vᵢₙₛₚ, Vₑₓₚ). Observation periods are in this case, for example, a breathing cycle, a plurality of breathing cycles, parts of breathing cycles such as inspiration, inspiratory pause, expiration, expiratory pause. The ventilator 40 is preferably in an operating state, in which it carries out a variation of the end-expiratory pressure (PEEP=Positive End Expiratory Pressure). For example, a so-called decremental PEEP trial is carried out by the ventilator 40 for this. In this connection, the end-expiratory pressure is reduced in steps from a starting level of, for example, 14 mbar to an end value of 6 mbar. These PEEP levels here have each, for example, a pressure difference of 2 mbar. By means of a synchronizing 44 of the introduction of the PEEP levels by the ventilator 40 with the data acquisition of the EIT data 36 carried out by the measured value acquisition and feed unit 32, it is possible to obtain sequences of tidal images and of differential tidal images 7 derived from these tidal images at defined pressure situations. This makes it possible to determine the lung regions Ω 2 (FIG. 1), Ω+ 4 (FIG. 1), Ω− 3 (FIG. 1) and the corresponding difference parameters 5, 300, 400 (FIG. 1) in a reproducible manner and thus to enable the user to monitor the state of the patient 35 in the course of ventilation therapy by the ventilator in a high-quality and comprehensible manner and also to make visible in a quantitative manner recovery progress with respect to the ventilation of the lungs of the patient 35 based on the difference parameters 300, 400 (FIG. 1).

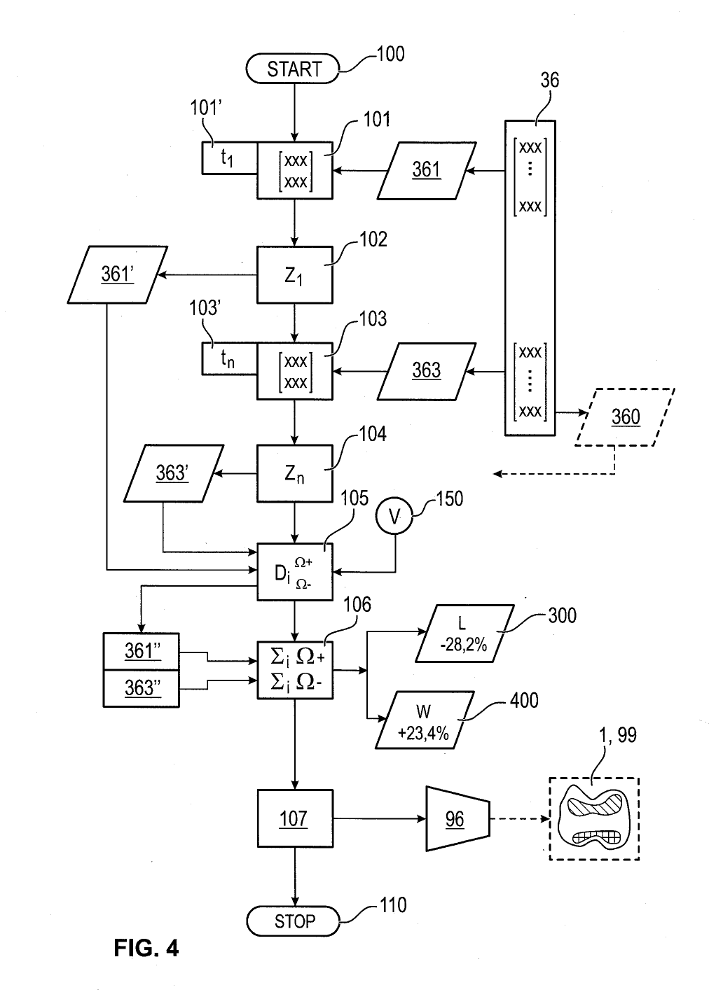

FIG. 4 shows in a schematic view a flow chart for determination of difference parameters 300, 400 on the basis of EIT data 36 as a sequence of steps 101, 102, 103, 104, 105, 106, 107, beginning from a starting time 100 up to an end time 110. The difference parameters L 300, W 400 indicate ventilation situations of regions Ω 2, Ω− 3, Ω+ 4 (FIG. 1) of the lungs of a patient 35 (FIG. 3), as explained with regard to FIG. 1.

EIT data 36, 361, which are provided and associated with a first time t₁ 101′, are inputted in the first step 101. This step 101 is carried out, for example, during or immediately after a measured value acquisition at the time t₁ 101′ of EIT data 36 by an EIT apparatus 30 (FIG. 3).

Regional impedance values Z₁ of ventilated lung regions are each determined on the basis of the EIT data 361 associated with the first time t₁ 101′ and are provided as a data set 361′ in the second step 102. The regional impedance values Z₁ overall produce in this case a reference tidal image of the lungs, which is used as a reference for determination of the differential tidal image 1 (FIG. 1) or of a plurality of differential tidal images in the fifth step 105.

EIT data 36, 363 provided and associated with an additional time tₙ 103′ are inputted in the third step 103. This step 103 is carried out, for example, in the course of an EIT apparatus performing the electrical impedance tomography during the measured value acquisition of EIT data 36 by an EIT apparatus 30 (FIG. 3) at the time tₙ. It is also possible that additional partial steps at times t_{n±m}, in which provided EIT data 36 are inputted, are carried out chronologically before the third step 103 or chronologically after the third step 103. These partial steps are not shown in FIG. 4 for the sake of clarity; with the drawing element 360 shown in dotted lines, this FIG. 4 only illustrates that additional EIT data 360 associated and provided at defined times can be inputted on the basis of the EIT data 36, which can then likewise be processed to form difference parameters L 300, W 400, as explained in connection with the subsequent steps 104, 105, 106, 107.

Regional impedance values Zₙ of ventilated lung regions, which from the basis of pixel values, are each determined/received on the basis of the EIT data 363 associated with the at least one additional time tₙ 101′ and are provided as a data set 363′ in the fourth step 104. The regional impedance values Zₙ overall produce in this case a current tidal image of the lungs.

Regional difference values 361″, 363″ of the ventilated lung regions Ω 2 (FIG. 1) are determined each on the basis of the data sets 361′, 363′ with the determined regional impedance values Z₁, Zₙ and are classified on the basis of an evaluation criterion V 150 in the fifth step 105. As noted, the determination of the regional impedance values Z₁, Zₙ, in the simplest form, involves receiving pre-processed data, for example an EIT-data set from an EIT system with a structure of pixels, each pixel having certain point in a coordinate system (Cartesian or Polar) in the two-dimensional horizontal layer (transverse view of the thorax) and a certain point in time (t1). With the simple determination there is no special data processing needed between steps 361 and 101. Step 101 only relocates the information related to the coordinate system into regional impedances 361′. In another way of implementation of the determination, the EIT-data set 36 contains an amount of data, for example a large matrix Z1, X1, Y1 . . . Zn, Xn, Yn with pixels and associated time information t1 . . . tn over a time period of monitoring a patient while changing the PEEP step by step. The steps 101 and 103 perform an extracting of the two certain data sets 361, 363 and then in steps 102, 104 regional impedances 361′, 363′ are carried out as outputs of these steps 102, 104. The regional difference values 361″, 363″ overall then result in a differential tidal image 1 (FIG. 1) with ventilated regions Ω 2 (FIG. 1) of the lungs with regions of positive difference values Ω+ 4 (FIG. 1) and regions of negative difference values Ω− 3 (FIG. 1). The determination of the difference values is carried out such that the reference tidal image, in the form of pixels, is subtracted from the current tidal image, in the form of pixels. The regional difference values are classified into positive and negative difference values. An adding up of all positively classified difference values of this differential tidal image results in a positive difference parameter W 400 (WIN). An adding up of all negatively classified difference values of this differential tidal image results in a negative difference parameter L 300 (LOSS). In this case, the classification is carried out such that negative difference values for pixels result when the regional impedance value Zₙ at the time tₙ is increased by a first predefined value compared to the regional impedance value Z₁ at the time t₁ and positive difference values for pixels result when the regional impedance value Zₙ at the time tₙ is lower by a second predefined value compared to the regional impedance value Z₁ at the time t₁. In this connection, for example, threshold values can be used as predefined values, for example, such that a difference in amount of 5%-15% is used with reference to the regional impedance value Z₁ at the time t₁. In this case, positive difference values indicate improvement, negative difference values indicate worsening of the ventilation situation in the lung regions in question.

The classified difference values 361″, 363″ are added up and the difference parameters L 300 (LOSS) and W 400 (WIN) are determined from the totals of the classified difference values 361″, 363″ in the sixth step 106.

As already stated with regard to the third step 103, additional current tidal images can be acquired, for example, in the form of a series of measured value acquisitions by the EIT apparatus 30 (FIG. 3) and then additional current tidal images can be determined based on this, so that determination of a series of differential tidal images is thus also possible and determination of a series of difference parameters L 300 (LOSS) and W 400 (WIN) is thus possible as well. Such a series of difference parameters L 300 (LOSS) and W 400 (WIN) offers the advantage of being able to make available an estimation with regard to the therapy and recovery course of the patient, especially in connection with a change in (ventilation) pressure conditions in the lungs of the patient brought about in this context. Such a change in (ventilation) pressure conditions may be caused here by means of a change in an inspiration pressure or in the pressure (PEEP=Positive End Expiratory Pressure), which is given at the time of an expiration phase in the lungs of a patient, carried out by the user. Such a change may, however, for example, also be caused by means of a maneuver carried out by a ventilator 40 (FIG. 4). Such a maneuver is configured, for example, as an incremental or decremental PEEP trial, in which the positive end-expiratory pressure (PEEP) is gradually increased or decreased from a starting value.

The classified difference values 361″, 363″ are added up separately here for regions of positive difference values Ω+ 4 (FIG. 1) and regions of negative difference values Ω− 3 (FIG. 1) to form the difference parameters L 300, W 400. The difference parameters L 300 (LOSS), W 400 (WIN) thus indicate regional property changes in at least two regional zones of the lungs, which are different from one another.

A control signal 96, which can be used as an output signal 96′ (FIG. 3), for example, for a visualization 99 in a differential tidal image 1 (FIG. 1), is generated and provided from the difference parameters 361″, 363″ in the seventh step 107.

While specific embodiments of the invention have been shown and described in detail to illustrate the application of the principles of the invention, it will be understood that the invention may be embodied otherwise without departing from such principles.

### APPENDIX

List of Reference Numbers

| | |
|---|---|
| 1 | Differential tidal image |
| 2 | Ventilated lung region |
| 3 | Lung region with loss (LOSS) − |
| 4 | Lung region with win (WIN) + |
| 5 | Percentage of WIN and LOSS values |
| 6 | Diagram of WIN and LOSS values |
| 7 | Course of an incremental PEEP trial |
| 8 | Pressure levels |
| 8′ | Reference pressure level |
| 10 | EIT device and electrode array |
| 30 | EIT apparatus |
| 32 | Measured value acquisition and signal feed unit |
| 34 | Torso, thorax, chest, chest region |
| 35 | Patient |
| 36, 360, 361, 363 | EIT data |
| 361′, 363′ | Data sets with regional impedance values Z |
| 40 | Ventilator, anesthesia apparatus |
| 44 | Pressure values, data of the ventilator |
| 50 | Data input unit |
| 55 | Data signal |
| 70 | Control unit |
| 77 | Memory |
| 79 | Data interface |
| 90 | Data output unit |
| 95 | Display device |
| 96 | Data signals, control signals |
| 96′ | Output signal |
| 99 | Visualization |
| 99′ | Operating elements, indicator elements |
| 100, 101-107, 110 | Start, sequence of steps, stop, end |
| 101′, 103′ | Times t₁, tₙ |
| 150 | Evaluation criterion V |
| 300, 400 | Difference parameters (WIN, LOSS) |

1. A device for determination of difference parameters based on electrical impedance tomography (EIT) data, the device comprising:
a data input unit, receiving the EIT data obtained by means of an electrical impedance tomography apparatus, wherein the data input unit is configured to receive and provide EIT data from at least one region of the lungs of a living being over an observation period;
an output unit;
a calculation and control unit connected to the output unit and connected to the data input unit, wherein the calculation and control unit is configured to determine regional impedance values at a first time from the EIT data for at least two regional zones of the lungs;
to determine additional regional impedance values at at least one additional time for the at least two regional zones of the lungs, the at least one additional time, as a further time, chronologically succeeding the first time;
to determine a regional difference value between the impedance value at the first time and the impedance value at the additional time for each of the at least two regional impedance values;
to classify difference values from the regional difference values determined on the basis of an evaluation criterion;
to add up the classified difference values and to determine difference parameters therefrom, which indicate regional property changes in the at least two regional zones of the lungs and to generate and provide a control signal from the difference parameters, wherein:
the output unit is configured to use the control signal to provide or output an output signal, which represents a numerical value of the regional property changes of each of the at least two regional zones of the lungs.

2. A device in accordance with claim 1, wherein:
the data input unit is configured to receive and provide data, which indicate measured pressure values or a pressure course of a ventilation pressure, of a ventilation course, in the observation period;
values, which indicate end-expiratory values of a ventilation pressure, are determined by the calculation and control unit on the basis of the data, which values indicate the pressure course or the measured pressure values of the ventilation course.

3. A device in accordance with claim 2, wherein the calculation and control unit:
determines regional end-expiratory impedance values of the regional zones of the lungs;
determines regional difference values from the regional end-expiratory impedance values; and
determines the difference parameters in each case taking into consideration the values, which indicate end-expiratory values of the ventilation pressure, from the regional impedance values.

4. A device in accordance with claim 1, wherein a duration of at least two breathing cycles, each with an inspiratory period and an expiratory period, is selected to be the observation period.

5. A device in accordance with claim 2, wherein:
a time period of a maneuver with changes in the ventilation pressure, in a decremental PEEP trial with a plurality of breathing cycles, each with a phase of inspiration and with a phase of expiration, is selected as the observation period; and
the end-expiratory ventilation pressure, corresponding to the regional end-expiratory impedance values, is lower by a predefined value at the further time compared to the end-expiratory ventilation pressure at the first time.

6. A device in accordance with claim 5, wherein the output unit, using the control signal, is configured to generate, provide or output the output signal, which represents the difference parameters as a function of the pressure-time course of the decremental PEEP trial or as a function of a time course of the decremental PEEP trial.

7. A device in accordance with claim 2, wherein:
a time period of a maneuver with changes in the ventilation pressure, in an incremental PEEP trial with a plurality of breathing cycles, each with a phase of inspiration and with a phase of expiration, is selected to be the observation period; and
the end-expiratory ventilation pressure (PEEP) corresponding to the regional end-expiratory impedance values is increased by a predefined value at the further time compared to the end-expiratory ventilation pressure (PEEP) at the first time.

8. A device in accordance with claim 7, wherein the output unit, using the control signal, is configured to generate, provide or output the output signal, which represents the difference parameters as a function of the pressure-time course of the incremental PEEP trial or as a function of a time course of the incremental PEEP trial.

9. A device in accordance with claim 1, wherein:
at least one rear lung region, a dorsal region of the lungs, is also included in a region of the at least two regional zones of the lungs; and
the calculation and control unit is configured to determine, from the regional impedance values, regional end-expiratory impedance values of the rear region of the lungs, to determine the regional difference values from the end-expiratory impedance values of the rear region of the lungs and to determine a difference parameter, which indicates property changes in the rear, dorsal region of the lungs.

10. A device in accordance with claim 9, wherein:
the property changes in the rear, dorsal region of the lungs relates to a win/loss situation of the regional changes in compliance in the dorsal region of the lungs;
the output unit, using the control signal, is configured to generate, provide or output the output signal, which represents numerical win/loss situations of the regional changes in compliance in the dorsal region of the lungs.

11. A device in accordance with claim 1, wherein:
at least one front region, a ventral region of the lungs, is also included in a region of the at least two regional zones of the lungs; and
the calculation and control unit is configured to determine, from the regional impedance values, regional end-expiratory impedance values of the front region of the lungs, to determine the regional difference values from the end-expiratory impedance values of the front region of the lungs and to determine a difference parameter, which indicates the property changes in the front, ventral region of the lungs.

12. A device in accordance with claim 11, wherein:
the property changes in the rear, ventral region of the lungs relates to a win/loss situation of the regional changes in compliance in the ventral region of the lungs;
the output unit, using the control signal, is configured to generate, provide or output the output signal, which represents numerical win/loss situations of the regional changes in compliance in the ventral region of the lungs.

13. A device in accordance with claim 1, wherein the data, which indicate a pressure course of a ventilation pressure or in the observation period, are provided to the device by a ventilator or an anesthesia apparatus and are inputted or provided by the data input unit and are processed by the calculation and control unit for determination of the difference parameters.

14. A device in accordance with claim 1, further comprising a display connected to the output unit for visualization of properties of lungs based on the EIT data for determination and visualization of properties of the lungs or property changes.

15. A device in accordance with claim 14, in combination with an apparatuses for carrying out ventilation or for carrying out anesthesia or for carrying out both ventilation and for carrying out anesthesia.

16. A method for determination of difference parameters based on electrical impedance tomography (EIT) data, the method comprising:
providing EIT data associated with a first time;
determining first time regional impedance values based on the EIT data associated with the first time and providing the first time regional impedance values as a first time regional impedance values data set;
providing EIT data associated with a further time;
determining further time regional impedance values based on the EIT data associated with the further time and are providing the further time regional impedance values as a further time regional impedance values data set;
determining regional difference values of ventilated lung regions based on the first time regional impedance values data set and the further time regional impedance values data set;
classifying the determined regional difference values based on an evaluation criterion;
adding up the classified difference values and determining difference parameters from totals of the classified difference values; and
generating a control signal from the difference parameters.

17. A method in accordance with claim 16, wherein additional EIT data are provided and are processed to form additional difference parameters.

18. A method in accordance with claim 17, wherein the additional difference parameters are determined based on a change in a ventilation pressure situation.

19. A method in accordance with claim 18, wherein the change in ventilation pressure situation is configured as a gradual change in an expiratory pressure, each at an end of expiration phases.
